(19) United States
(12) Patent Application Publication
(10) Pub. No.: US 2017/0225100 A1
(43) Pub. Date: Aug. 10, 2017

(54) DISTILLATION DEVICE

(71) Applicant: LG CHEM, LTD., Seoul (KR)

(72) Inventors: Yeon Uk CHOO, Daejeon (KR); Sung Kyu LEE, Daejeon (KR); Tae Woo KIM, Daejeon (KR); Joon Ho SHIN, Daejeon (KR)

(21) Appl. No.: 15/500,365

(22) PCT Filed: Jun. 30, 2016
(86) PCT No.: PCT/KR2016/007021

§ 371 Date: Jan. 30, 2017

(30) Foreign Application Priority Data
Jun. 30, 2015 (KR) 10-2015-0093695

(51) Int. Cl. B01D 3/32; C07C 7/04; C07C 45/84; B01D 3/34; B01D 3/14

(52) U.S. Cl. CPC B01D 3/32; B01D 3/34; B01D 3/141; C07C 45/84; C07C 7/04

(57) ABSTRACT

The present application relates to a distillation device, and according to the distillation device of the present application, in first and second compounds being capable of forming an azeotrope, by introducing the second compound having a relatively high boiling point into a supply port located below the first compound having a relatively low boiling point, the first compound can be previously separated from the top of a first distillation column and the content of the first compound in the flow discharged from the bottom of the first distillation column can be minimized, and thus, as a moving route of the first compound is minimized, the second compound can be separated in high purity.

The present application is a National Stage Application No. PCT/KR2016/007021, filed Jun. 30, 2016, and claims the benefit of priority based on Korean Patent Application No. 10-2015-0093695 dated Jun. 30, 2015, all of which are hereby incorporated by reference in their entirety for all purposes as if fully set forth herein.

### TECHNICAL FIELD

The present application relates to a distillation device.

### BACKGROUND ART

Phenol is used in various fields as feedstocks of various synthetic resins such as polycarbonate resin and epoxy resin, including phenol resin, or feedstocks in the pharmaceutical industry, and feedstocks of detergents, such as nonylphenol, or various color paints.

Methods for producing phenol from cumene are well known. For example, the cumene is oxidized using a gas containing oxygen to form cumene hydroperoxide, which is again decomposed under an acidic catalyst, thereby resulting in phenol and acetone.

In the process of generating phenol as above, various side reactions occur at the same time. For example, dimethylbenzyl alcohol (DMBA) is formed as a major by-product in the oxidation step and is dehydrated subsequently in the same acid catalytic cracking step to produce alpha-methylstyrene (AMS). Meanwhile, hydroxyacetone (HA) among the side reaction products affect most highly on purity of phenol.

Accordingly, a distillation method for separating the hydroxyacetone more effectively is required.

### DISCLOSURE

### Technical Problem

The present application is intended to provide a distillation device which separates hydroxyacetone and phenol at low cost and high purity.

### Technical Solution

One embodiment of the present application provides a distillation device. According to the exemplary distillation device of the present application, in a first compound and a second compound being capable of forming an azeotrope, by introducing the second compound having a relatively high boiling point into a supply port located below the first compound having a relatively low boiling point, the first compound can be previously separated from the top of a first distillation column and the content of the first compound in a flow discharged from the bottom of the first distillation column can be minimized, and thus as the moving route of the first compound is minimized, the second compound can be separated in high purity. In addition, since the used amount of a solvent, for example water, required for removing the first compound and impurities can be reduced in the upper portion of a third distillation column, which is a low boiling point component removal device, the energy saving effect can be maximized

Hereinafter, the distillation device of the present application will be described with reference to the attached drawings, but the attached drawings are illustrative, and the distillation device of the present application is not limited by the attached drawings.

FIG. 1 is a diagram schematically showing a distillation device according to one embodiment of the present application.

As in FIG. 1, the distillation device of the present application comprises at least one or more distillation units. The term “distillation unit” above means one unit body which comprises a distillation column and a condenser and a reboiler, connected to the distillation column, respectively, and can perform distillation processes.

The distillation column is a device being capable of separating multi-component materials contained in feedstocks by each boiling point difference. Distillation columns having various shapes can be used in the distillation device of the present application in consideration of boiling points of components of the introduced feedstocks or components to be separated. The specific type of the distillation column which can be used in the distillation device of the present application is not particularly limited, and for example, a distillation column having a general structure as shown in FIG. 1 or a dividing wall distillation column equipped with a dividing wall inside may be also used. In one example, the distillation column can be divided into an upper region and a lower region. The term “upper region” herein may mean a relatively upper portion in the structure of the distillation column, and for example, mean the uppermost portion of the divided two regions when the distillation column is divided into two portions in the height direction or the longitudinal direction of the distillation column In addition, the above “lower region” may mean a relatively lower portion in the distillation column structure, and for example, mean the downmost portion of the divided two regions when the distillation column is divided into two portions in the height direction or the longitudinal direction of the distillation column. Herein, the upper region and the lower region of the distillation column can be used in a relative concept to each other. The top of the distillation column is included in the upper region and the bottom of the distillation column is included in the lower region; however, unless otherwise defined herein, the upper region is used in the same sense as the top region and the lower region is used in the same sense as the bottom region. As the distillation column, a distillation column having a number of theoretical stages of 32 to 98 can be used. In the above, the “number of theoretical stages” means a number of imaginary regions or stages in which two phases such as a vapor phase and a liquid phase in the distillation column are in equilibrium with each other.

In one embodiment, as in FIG. 1, the first distillation unit (10) comprises a first distillation column (100), and a first condenser (110) and a first reboiler (120), connected to the first distillation column (100), respectively. For example, the first distillation column (100), the first condenser (110), and the first reboiler (120) may be fluidically connected to each other so that the fluid introduced into the first distillation column (100) can flow. The “condenser” above is a device separately installed outside the distillation column, and means a device for cooling the flow discharged from the top of the distillation column by a method such as contacting it with cooling water introduced outside. For example, the first condenser (110) of the first distillation column (100) is a device for condensing a first top flow (Fₜₒₚ₁) discharged from the top region of the first distillation column (100), and a second condenser (210) and a third condenser (310) of a second distillation column (200) and a third distillation column (300), which are described below, may be devices for condensing a second top flow (Fₜₒₚ₂) discharged from the top region of the second distillation column (200) and a third top flow (Fₜₒₚ₃) discharged from the top region of the third distillation column (300). In addition, the reboiler” above may be a heating device separately installed outside the distillation column and mean a device for again heating and evaporating a flow of high-boiling components discharged from the bottom of the distillation column. For example, the first reboiler (120) of the first distillation column (100) is a device for heating a first bottom flow (F_{btm1}) discharged from the bottom region of the first distillation column (100), and a second reboiler (220) of the second distillation column and a third reboiler (320) of the third distillation column (300), which are described below, may be devices for heating a second bottom flow (F_{btm2}) discharged from the bottom region of the second distillation column (200) and a third bottom flow discharged from the bottom region of the distillation column (300).

The first distillation column (100) comprises a first supply port (101) and a second supply port (102) located below the first supply port (101). In one embodiment, when the first distillation column (100) is divided into an upper region and a lower region, the first supply port (101) may be located at the upper region of the first distillation column (100), and the second supply port (102) may be located at the lower region of the first distillation column (100). In another embodiment, both the first supply port (101) and the second supply port (102) may be located at the upper region of the first distillation column (100), where the first supply port (101) may be located above the second supply port (102), for example, at the upper stage. In one example, the first supply port (101) may be located at 1 to 40% of the number of theoretical stages calculated based on the top. In addition, the second supply port (102) may be located at 40 to 100% of the number of theoretical stages calculated based on the top. For example, when the number of theoretical stages of the distillation column is 100 stages, the first stage of the distillation column corresponds to the top and the 100th stage corresponds to the bottom, where the first supply port (101) can be located at the 1st to 40th stages and the second supply port (102) can be located at the 40th to 100th stages.

As shown in FIG. 1, the feedstock (F₁) containing the first compound flows into the first supply port (101) of the first distillation column (100), and the feedstock (F₂) containing the second compound forming an azeotrope with the first compound flows into the second supply port (102).

The first compound and the second compound are not particularly limited as long as they are mixed with each other to form an azeotrope. The term “azeotrope” above means a liquid mixture in a solution state in which azeotropy or the like may occur. Generally, if a solution is distilled, the composition changes according to boiling, with usually raising or lowering the boiling point as well, but a certain type liquid having a special ratio of components boils without changing the ratio of components at a certain temperature like a pure liquid, where the ratios of components in solution and vapor become same, and then the system is referred to as being in an azeotropic state, the ratio of components is referred to as an azeotropic composition, the solution is referred to as an azeotrope and the boiling point of the azeotrope is referred to as an azeotropic point. In one example, the first compound may be hydroxyacetone, and the second compound being capable of forming an azeotrope with the hydroxyacetone may be alpha-methylstyrene, without being particularly limited thereto.

In the distillation device of the present application, the first and second compounds being capable of forming an azeotrope with each other are introduced at different positions of the distillation column, and in particular, the second compound having a relatively high boiling point of the first and second compounds being capable of forming the azeotrope is introduced into the supply port located below the first compound having a relatively low boiling point, and thus the first compound may be previously separated from the top of the first distillation column (100) and the content of the first compound in the flow discharged from the bottom of the distillation column (100) may be minimized, whereby the content of the first compound separated from the second distillation column (200) and the third distillation column (300), which are described below, may be minimized That is, according to the distillation device of the present application, as the moving route of the first compound is minimized, the second compound can be separated to high purity and the energy saving effect can be maximized.

In one example, the feedstocks (F₁, F₂) containing the first and second compounds introduced into the first supply port and the second supply port (102) of the first distillation column (100), respectively, are divided to the first top flow (Fₜₒₚ₁) discharged from the top region of the first distillation column (100) and the first bottom flow (F_{btm1}) discharged from the bottom region of the first distillation column (100), respectively, and discharged. The first top flow (Fₜₒₚ₁) discharged from the top region of the first distillation column (100) flows into the first condenser (110) and some or all of the first top flow (Fₜₒₚ₁) passing through the first condenser (110) may be refluxed to the top region of the first distillation column (100) or stored as a product. In one example, the flow discharged from the first condenser (110) flows into a storage tank and is stored, and then can be refluxed to the first distillation column (100) or stored as a product. In addition, a portion of the first bottom flow (F_{btm1}) discharged from the bottom region of the first distillation column (100) may flow into the first reboiler (120), a portion of the first bottom flow (F_{btm1}) passing through the first reboiler (120) may be refluxed to the bottom region of the first distillation column (100) and the remaining portion may flow into the second distillation column to be described below.

In one embodiment, the first top flow (Fₜₒₚ₁) comprises a relatively low boiling point component of feedstock (F₁, F₂) components introduced into the first distillation column (100), and in one example, it comprises the first compound, the second compound, and a substance having a boiling point lower than that of the second compound. In addition, the first bottom flow (F_{btm1}) comprises a relatively high boiling point component among the components contained in the feedstocks (F₁, F₂) introduced into the first distillation column (100) and in one example, it comprises the first compound and a substance having a boiling point higher than that of the first compound. In one example, as described above, the first compound may be hydroxyacetone, where the second compound may be alpha-methylstyrene and the substance having a boiling point lower than that of the second compound may comprise one or more selected from acetone, cumene and water, without being limited thereto. Furthermore, the substance having a boiling point higher than that of the first compound may comprise one or more selected from the group consisting of cumene, phenol, and methylphenyl ketone, but is not limited thereto. In one embodiment, when the boiling point of the second compound is higher than that of the first compound, the first top flow (Fₜₒₚ₁) may be a flow that a concentration of the first compound is relatively higher than that of the second compound, and the first bottom flow (F_{btm1}) may be a flow that a concentration of the first compound is relatively lower than that of the second compound.

In the distillation device of the present application, as described above, the second compound having a relatively high boiling point, among the first and second compounds being capable of forming the azeotrope, is introduced into the supply port located below the first compound having a relatively low boiling point, and thus the first compound can be previously separated from the top of the first distillation column (100) and the content of the first compound in the flow discharged from the bottom of the first distillation column (100) can be minimized In one example, the content of the first compound in the first bottom flow (F_{btm1}) may be 0.005 to 0.25 parts by weight, for example, 0.01 to 0.03 parts by weight, relative to 100 parts by weight of the total components contained in the first bottom flow (Fbₜₘ₁). By controlling the content of the first compound in the first bottom flow (F_{btm1}) within the above range, the content of the first compound separated in the second distillation column (200) and the third distillation column (300), which are described below, can be minimized, and as the moving route of the first compound is minimized, the second compound can be separated in high purity and the energy saving effect can be maximized

In one example, when the content of the first compound in the first bottom flow (F_{btm1}) of the first distillation column (100) is controlled within the above range, the content of the first compound in the first top flow (Fₜₒₚ₁) of the first distillation column (100) may be 0.01 to 2.0 parts by weight, for example, 0.1 to 0.5 parts by weight, relative to 100 parts by weight of the total components contained in the first top flow (Fₜₒₚ₁).

In the unique distillation device of the present application in which a flow (F₁) of the feedstock containing the above mentioned first compound flows into the first supply port (101) and a flow (F₂) of the feedstock containing the second compound being capable of forming an azeotrope with the first compound flows into the second supply port (102) located below the first supply port (101), another embodiment of the present application provides design conditions of the distillation device optimized in the above distillation device. In one example, the temperature of the feedstock (F₂) comprising the second compound introduced into the second supply port (102) may be from 20 to 180° C., for example from 23 to 25° C., or from 168 to 172° C. In addition, the flow rate of the feedstock (F₂) containing the second compound introduced into the second supply port (102) may be 300 to 1200 kg/hr, for example, 400 to 600 kg/hr, or 900 to 1100 kg/hr.

As in FIG. 1, the distillation device of the present application may further comprise a second distillation unit (20) and a third distillation unit (30) in addition to the above mentioned first distillation unit (10).

In one embodiment, the distillation device may further comprise the second distillation unit (20) and the third distillation unit (30), where the second distillation unit (20) may comprise a second condenser (210), a second reboiler (220) and a second distillation column (200) and the third distillation unit (30) may comprise a third condenser (310), a third reboiler (320) and a third distillation column (300).

A portion of the first bottom flow (F_{btm1}) discharged from the bottom of the first distillation column (100) may flow into the second distillation column (200). In addition, the flow introduced into the second distillation column (200) may be divided into a second top flow (Fₜₒₚ₂) discharged from the top region of the second distillation column (200) and a second bottom flow (F_{btm2}) discharged from the bottom region of the second distillation column (200), respectively, and discharged.

The second top flow (Fₜₒₚ₂) comprises a relatively low boiling point component among the components contained in the first bottom flow (F_{btm1}) introduced into the second distillation column (200), and in one example, it may comprise one or more selected from hydroxyacetone, alpha-methylstyrene, phenol and 2-methylbenzofuran, but is not limited thereto. In addition, the second bottom flow (F_{btm2}) comprises a relatively high boiling point component among the components contained in the first bottom flow (F_{btm1}) introduced into the second distillation column (200), and in one example, it may comprise methylphenyl ketone, dicumyl peroxide, and p-cumylphenol, but is not limited thereto.

The second top flow (Fₜₒₚ₂) discharged from the second top region may flow into the third distillation column (300). In addition, the flow introduced into the third distillation column (300) can be divided into a third top flow (Fₜₒₚ₃) discharged from the top region of the third distillation column (300) and a third bottom flow discharged from the bottom region of the third distillation column (300), respectively, and discharged. The third top flow (Fₜₒₚ₃) comprises a relatively low boiling point component among the components contained in the second top flow (Fₜₒₚ₂) introduced into the third distillation column (300), and in one example, it may comprise one or more selected from the group consisting of hydroxyacetone, alpha-methylstyrene and 2-methylbenzofuran, but is not limited thereto. In the distillation device of the present application, as described above, by controlling the content of the first compound in the first bottom flow (F_{btm1}) within a specific range, the content of the first compound separated from the second distillation column (200) and the third distillation column (300) can be minimized. In one example, the content of the first compound, e.g., hydroxyacetone, in the third top flow (Fₜₒₚ₃) may be controlled to be included in a very low amount, and for example, it may be 0.01 to 5.0 parts by weight, relative to 100 parts by weight of the total components contained in the third top flow (Fₜₒₚ₃), but is not limited thereto.

The third bottom flow comprises a relatively high boiling point component among the components contained in the second top flow (Fₜₒₚ₂) introduced into the third distillation column (300), and in one example, it may comprise one or more selected from the group consisting of phenol, and water, but is not limited thereto. In one embodiment, the third bottom flow may be a flow of pure phenol.

Hereinafter, the process of separating phenol and hydroxyacetone using the distillation device according to one embodiment of the present application will be described in more detail.

In one example, a feedstock (F₁) containing hydroxyacetone and phenol flows into the first supply port (101) of the first distillation column (100), and a feedstock (F₂) containing alpha-methylstyrene being capable of forming an azeotrope with the hydroxyacetone flows into the second supply port (102) located below the first supply port (101) of the first distillation column (100).

In this case, the flow that pure acetone is rich, which is a relatively low boiling point component among the components contained in the feedstock (F₁) introduced into the first supply port (101), may flow out of the top region of the first distillation column (100) as the first top flow (Fₜₒₚ₁), and the flow that phenol is rich, which is a relatively high boiling point component, may flow out of the bottom region of the first distillation column (100) as the first bottom flow (F_{btm1}). The first top flow (Fₜₒₚ₁) discharged from the top region of the first distillation column (100) may pass through the first condenser (110) to reflux to the top region of the first distillation column (100), and the remaining portion may be stored as a product. The product may be pure acetone in high purity. The first top flow (Fₜₒₚ₁) may contain some cumene, alpha-methylstyrene and hydroxyacetone in addition to acetone, and as described above, the content of hydroxyacetone in the first top flow (Fₜₒₚ₁) may be 0.01 to 2.0 parts by weight relative to 100 parts by weight of the total components contained in the first top flow (Fₜₒₚ₁).

Moreover, a portion of the first bottom flow (F_{btm1}) discharged from the bottom region of the first distillation column (100) may pass through the first reboiler (120) for some to be refluxed to the bottom region of the first distillation column (100) and for the remaining portion to flow into the second distillation column (200). In addition, the flow that phenol is rich, which is a relatively low boiling point component among the components contained in the feedstock flow introduced into the second distillation column (200), may flow out of the top region of the second distillation column (200) as the second top flow (Fₜₒₚ₂), and the flow that methylphenyl ketone with a relatively high boiling point is rich, may flow out of the bottom of the second distillation column (200) as the second bottom flow (F_{btm2}). The discharged second top flow (Fₜₒₚ₂) may flow into the storage tank via the second condenser (210) for a portion of the flow discharged from the storage tank to be refluxed to the top region of the second distillation column (200) and for the remaining portion to flow into the third distillation column (300). In addition, the high boiling point flow having a relatively high boiling point among the components contained in the flow introduced into the second distillation column (200) may flow out of the bottom of the second distillation column (200) as the second bottom flow (F_{btm2}) for a portion of the second bottom flow (F_{btm2}) to be refluxed to the bottom region of the second distillation column (200) via the second reboiler (220) and for the remaining portion to be stored as a product. The product may be methylphenyl ketone in high purity.

The second top flow (Fₜₒₚ₂) discharged from the top region of the second distillation column (200) may flow into the third distillation column (300). The flow that alpha-methylstyrene is rich, which is a relatively low boiling point component among the components contained in the second top flow (Fₜₒₚ₂) introduced into the third distillation column (300), may flow out of the top region of the third distillation column (300) as the third top flow (Fₜₒₚ₃), the third top flow (Fₜₒₚ₃) discharged from the top region of the third distillation column (300) may pass through the third condenser (310) to be refluxed to the top region of the third distillation column (300), and the remaining portion can be stored as a product. The product may be alpha-methylstyrene in high purity. In this case, the content of hydroxyacetone in the third top flow (Fₜₒₚ₃) may be adjusted to a very small range, and for example, the content of hydroxyacetone may be 0.01 to 5.0 parts by weight, relative to 100 parts by weight of the components contained in the third top flow (Fₜₒₚ₃). In addition, the high boiling point flow having a relatively high boiling point among the components contained in the flow introduced into the third distillation column (300) may flow out of the bottom region of the third distillation column (300) as the third bottom flow (F_{btm3}) for a portion of the third bottom flow (F_{btm3}) to be refluxed to the bottom region of the third distillation column (300) via the third reboiler (320) and for the remaining portion to be stored as a product. The product may be phenol in high purity.

The term “low boiling point flow” herein means a flow in which a relatively low boiling point component among the feedstock flow comprising low boiling point and high boiling point components is rich, and the low boiling point flow means, for example, a flow discharged from the top region of the first distillation column (100), the second distillation column (200) and the third distillation column (300). Also, the “high boiling point flow” means a flow in which a relatively high boiling point component among the feedstock flow comprising low boiling point and high boiling point components is rich, and the high boiling point flow means, for example, a flow that a relatively high boiling point component is rich, discharged from the bottom region of the first distillation column (100), the second distillation column (200) and the third distillation column (300). The term “rich flow” in the above means the flow having each content of low boiling point components contained in the flow discharged from the top region of the first distillation column (100), the second distillation column (200) and the third distillation column (300) and high boiling point components contained in the flow discharged from the bottom region of the first distillation column (100), the second distillation column (200) and the third distillation column (300), higher than each content of low boiling point components and high boiling point components contained in the feedstocks introduced into the first distillation column (100), the second distillation column (200) and the third distillation column (300), respectively. For example, it may means a flow that each content represented by the low boiling point component contained in the first top flow (Fₜₒₚ₁) of the first distillation column (100), the low boiling point component contained in the second top flow (Fₜₒₚ₂) of the second distillation column (200) and the low boiling point component contained in the third top flow (Fₜₒₚ₃) of the third distillation column (300) is at least 50% by weight, at least 80% by weight, at least 90% by weight, at least 95% by weight, or at least 99% by weight or mean a flow that each content represented by the high boiling point component contained in the first bottom flow (F_{btm1}) of the first distillation column (100) and the high boiling point component contained in the second bottom flow (F_{btm2}) of the second distillation column (200) and the high boiling point component contained in the third bottom flow (F_{btm3}) of the third distillation column (300) is at least 50% by weight, at least 80% by weight, at least 90% by weight, at least 95% by weight, or at least 99% by weight.

The present application also provides the above distillation method. An exemplary distillation method of the present application can be carried out using the above-described distillation device, and accordingly, the contents overlapping with those described in the above-mentioned distillation device will be omitted.

The preparation method of the present application comprises a feedstock supply step and a first distillation step.

In one embodiment, the feedstock supply step comprises i) introducing a feedstock (F₁) comprising the first compound into the first supply port (101) of the first distillation column (100), and ii) introducing a feedstock (F₂) comprising the second compound forming an azeotrope with the first compound 1 into the second supply port (102) located below the first supply port (101) and located at 40 to 100% of the number of theoretical stages calculated on the basis of the top. In addition, the first distillation step comprises iii) discharging the feedstock comprising the first and second compounds introduced into the first supply port and the second supply port (102) as the first top flow (Fₜₒₚ₁) discharged from the top region of the first distillation column (100) and the first bottom flow (F_{btm1}) discharged from the bottom region of the first distillation column (100).

Since the steps i) and ii) of the feedstock supply step and the step iii) of the first distillation step are each independently organically bonded, each boundary is not clearly divided according to the order of time, and thus the respective steps of i) to iii) may be performed sequentially or each independently at the same time.

In one embodiment, the first top flow (Fₜₒₚ₁) comprises the first compound, the second compound and a substance having a boiling point lower than that of the second compound, and the first bottom flow (F_{btm1}) comprises the first compound and a substance a boiling point higher than that of the first compound, and detailed descriptions thereof will be omitted since they are the same as those described in the above-mentioned distillation device.

In addition, the content of the first compound in the first bottom flow (F_{btm1}) may be 0.005 to 0.25 parts by weight, for example, 0.01 to 0.03 parts by weight, relative to 100 parts by weight of the total components contained in the first bottom flow (F_{btm1}). By controlling the content of the first compound in the first bottom flow (F_{btm1}) within the above range, the content of the first compound separated in the second distillation column (200) and the third distillation column (300), which are described below, can be minimized, and as the moving route of the first compound is minimized, the second compound can be separated in high purity and the energy saving effect can be maximized

In one example, when the content of the first compound in the first bottom flow (F_{btm1}) of the first distillation column (100) is adjusted within the above range, the first top flow (Fₜₒₚ₁) of the first distillation column (100) may be 0.01 to 2.0 parts by weight, for example, 0.1 to 0.5 parts by weight, relative to 100 parts by weight of the total components contained in the first top flow (Fₜₒₚ₁).

In the distillation method of the present application, the temperature of the first top flow (Fₜₒₚ₁) discharged from the top region of the first distillation column (100) may be 89° C. to 107° C., for example, 90° C. to 100° C. In addition, the temperature of the first bottom flow (F_{btm1}) discharged from the bottom of the first distillation column (100) may be 197° C. to 219° C., for example, 190° C. to 210° C.

In addition, in this case, the pressure of the top region of the first distillation column (100) may be 0.01 to 1.0 kgf/cm²g, for example, 0.1 to 0.5 kgf/cm²g. Also, the pressure of the bottom region of the first distillation column (100) may be 0.5 to 1.5 kgf/cm²g, for example, 0.5 to 1.0 kgf/cm²g.

In one example, the first compound may be hydroxyacetone, where the second compound may be alpha-methylstyrene and the substance having a boiling point lower than that of the second compound may comprise one or more selected from the group consisting of acetone, cumene and water, without being limited thereto. In addition, the substance having a boiling point higher than that of the first compound may comprise one or more selected from the group consisting of cumene, phenol, and methylphenyl ketone, but is not limited thereto.

In one example, the temperature of the feedstock (F₂) comprising the second compound introduced into the second supply port (102) may be 20 to 180° C., for example, 23 to 25° C., or 168 to 172° C. In addition, the flow rate of the feedstock (F₂) comprising the second compound introduced into the second supply port (102) may be 300 to 1200 kg/hr, for example, 400 to 600 kg/hr, or 900 to 1100 kg/hr.

### Advantageous Effects

According to the distillation device of the present application, by introducing the second compound having a relatively high boiling point, among the first and second compounds being capable of forming the azeotrope, into the supply port located below the first compound having a relatively low boiling point, the first compound can be previously separated from the top of the first distillation column and the content of the first compound in the flow discharged from the bottom of the first distillation column can be minimized, and thus as the moving route of the first compound is minimized, the second compound can be separated in high purity.

### BRIEF DESCRIPTION OF DRAWINGS

FIG. 1 is a diagram illustratively showing a distillation device according to one embodiment of the present application.

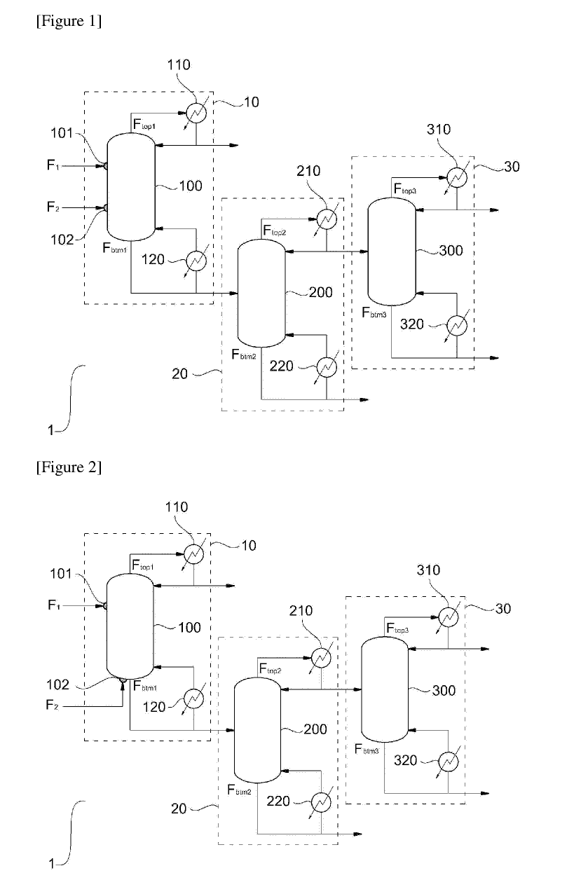

FIGS. 2 to 5 are diagrams schematically showing distillation devices used in Examples 1 to 4 of the present application.

FIG. 6 is a diagram illustratively showing a typical separation apparatus used in Comparative Example.

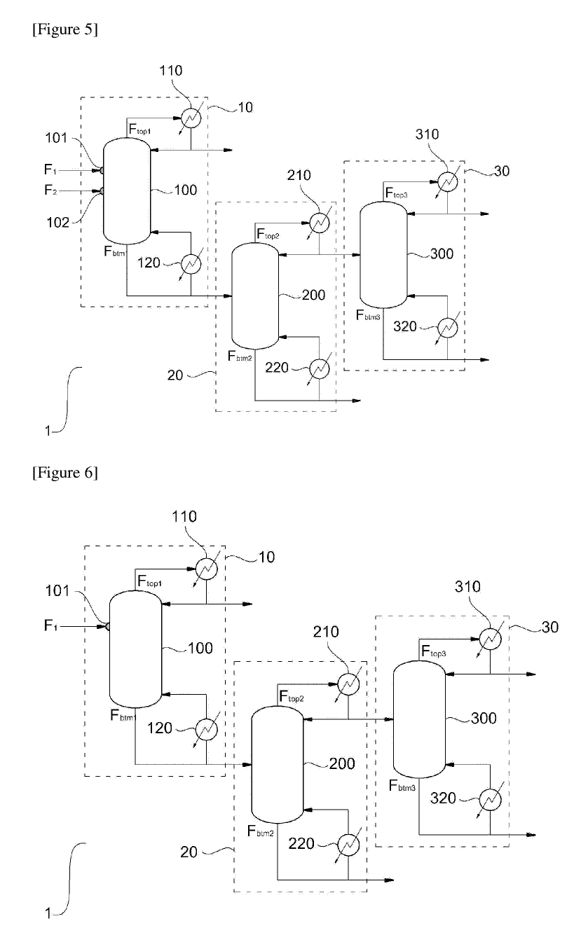

10: first distillation unit

100: first distillation column 101: first supply port

102: second supply port 110: first condenser

120: first reboiler 20: second distillation unit

200: second distillation column 210: second condenser

220: second reboiler 30: third distillation unit

300: third distillation column 310: third condenser

320: third reboiler F₁: feedstock containing the first compound

F₂: feedstock containing the second compound forming an azeotrope with the first compound

Fₜₒₚ₁: first top flow F_{btm1}: first bottom flow

Fₜₒₚ₂: second top flow F_{btm2}: second bottom flow

Fₜₒₚ₃: third top flow F_{btm3}: third bottom flow

### MODE FOR INVENTION

Hereinafter, the present invention will be described in more detail through Examples complying with the present invention and Comparative Example uncomplying with the present invention, but the scope of the present invention is not limited by the proposed examples.

### Example 1

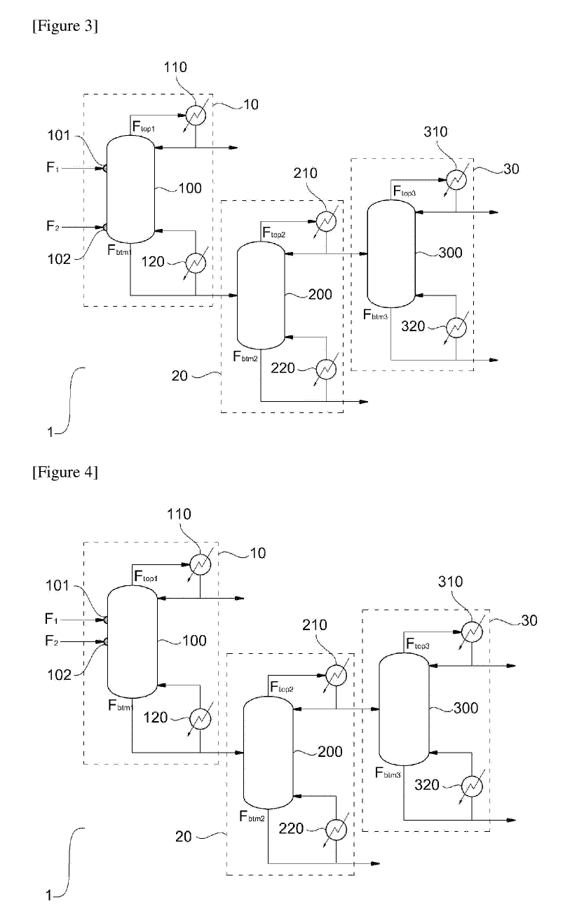

Phenol and hydroxyacetone were separated using the distillation device of FIG. 2.

Specifically, a feedstock containing 29% by weight of acetone, 9% by weight of cumene, 3% by weight of alpha-methylstyrene, 0.2% by weight of hydroxyacetone, 46% by weight of phenol and 3% by weight of a high boiling point component was introduced into the first supply port located at the 20th stage of the first distillation column having a number of theoretical stages of 65 at a temperature of 106° C. and a flow rate of 85,000 kg/hr. Furthermore, in addition to this, a feedstock containing 99.8% by weight of alpha-methylstyrene was introduced into the second supply port located at the 65th stage of the first distillation column at a temperature of 170.6° C. and a flow rate of 500 kg/hr.

The first top flow discharged from the top region of the first distillation column passed through the first condenser and a portion was refluxed to the top region of the first distillation column. The remaining portion of the first top flow was separated and stored as a product comprising 56% by weight of acetone, 17% by weight of cumene, 6% by weight of alpha-methylstyrene and 0.3% by weight of hydroxyacetone, and the first bottom flow discharged from the bottom region of the first distillation column passed through the first reboiler, and a portion was refluxed to the bottom region of the first distillation column and the remaining portion was introduced into the second distillation column In this case, the operating pressure of the first distillation column top region was adjusted to 0.2 kgf/cm²g, the operating temperature was adjusted to 94.1° C., the operating pressure of the first distillation column bottom region was adjusted to 0.716 kgf/cm²g, and the operating temperature was adjusted to be 203.1° C.

Furthermore, the second top flow discharged from the top region of the second distillation column passed through the second condenser, and a portion was refluxed to the top region of the second distillation column and the remaining portion was introduced into the third distillation column. A portion of the second bottom flow discharged from the bottom region of the second distillation column was refluxed to the bottom region of the second distillation column through the second reboiler and the remaining portion was separated as a product comprising 21% by weight of methylphenyl ketone and 20% by weight of p-cumylphenol. In this case, the operating pressure of the top region of the second distillation column was adjusted to −0.666 kgf/cm²g, the operating temperature was adjusted to be 147° C., the operating pressure of the bottom region of the second distillation column was −0.291 kgf/cm²g and the operating temperature was adjusted to be 213° C.

In addition, the third top flow discharged from the top region of the third distillation column passed through the third condenser, and a portion was refluxed to the top region of the third distillation column and the remaining portion was stored as a product comprising 0.11% by weight of hydroxyacetone and 68% by weight of alpha-methylstyrene. A portion of the third bottom flow discharged from the bottom region of the third distillation column was refluxed to the bottom region of the third distillation column via the third reboiler and the remaining portion was separated as a product containing pure phenol. In this case, the operating pressure of the top region of the third distillation column was adjusted to 0.03 kgf/cm²g, the operating temperature was adjusted to be 85° C., the operating pressure of the bottom region of the third distillation column was adjusted to 1.32 kgf/cm²g, and the operating temperature was adjusted to be 214° C.

In the case of separating phenol and hydroxyacetone using the distillation device of Example 1, the content of hydroxyacetone in the first bottom flow, the used amount of energy in the first and second reboilers, the amount of reduction, the rate of reduction and the purity of the phenol product were shown in Table 1 below.

### EXAMPLES 2 TO 10

Phenol and hydroxyacetone were separated by the same method as Example 1, except that the operating conditions of the first distillation column and the second distillation column were changed as in Table 1 below.

In the case of separating phenol and hydroxyacetone using the distillation devices of Examples 2 to 10, the content of hydroxyacetone in the first bottom flow, the used amount of energy in the reboilers, the amount of reduction, the rate of reduction and the purity of the phenol products were shown in Table 1 below.

### COMPARATIVE EXAMPLE

Phenol and hydroxyacetone were separated using the distillation device of FIG. 2.

Specifically, a feedstock comprising 29% by weight of acetone, 9% by weight of cumene, 3% by weight of alpha-methylstyrene, 0.2% by weight of hydroxyacetone, 46% by weight of phenol and 3% by weight of a high boiling point component was introduced into the first supply port located at the 20th stage of the first distillation column having a number of theoretical stages of 65.

The first top flow discharged from the top region of the first distillation column passed through the first condenser and a portion was refluxed to the top region of the first distillation column. The remaining portion of the first top flow was separated and stored as a product comprising 56% by weight of acetone, 17% by weight of cumene, 5% by weight of alpha-methylstyrene and 0.3% by weight of hydroxyacetone, and a portion of the first bottom flow discharged from the bottom region of the first distillation column was refluxed to the bottom region of the first distillation column via the first reboiler and the remaining portion flowed into the second distillation column. In this case, the operating pressure of the first distillation column top region was adjusted to 0.2 kgf/cm²g, the operating temperature was adjusted to 93.4° C., the operating pressure of the first distillation column bottom region was adjusted to 0.716 kgf/cm²g, and the operating temperature was adjusted to be 203.1° C.

Furthermore, the second top flow discharged from the top region of the second distillation column passed through the second condenser, and a portion was refluxed to the top region of the second distillation column and the remaining portion was introduced into the third distillation column. A portion of the second bottom flow discharged from the bottom region of the second distillation column was refluxed to the bottom region of the second distillation column through the second reboiler and the remaining portion was separated as a product. In this case, the operating pressure of the top region of the second distillation column was adjusted to −0.666 kgf/cm²g, the operating temperature was adjusted to be 147° C., the operating pressure of the bottom region of the second distillation column was −0.291 kgf/cm²g and the operating temperature was adjusted to be 213° C.

In addition, the third top flow discharged from the top region of the third distillation column passed through the third condenser, and a portion was refluxed to the top region of the third distillation column and the remaining portion was stored as a product comprising 1.08% by weight of hydroxyacetone. A portion of the third bottom flow discharged from the bottom region of the third distillation column was refluxed to the bottom region of the third distillation column via the third reboiler and the remaining portion was separated as a product containing pure phenol. In this case, the operating pressure of the top region of the third distillation column was adjusted to 0.03 kgf/cm²g, the operating temperature was adjusted to be 83° C., the operating pressure of the bottom region of the third distillation column was adjusted to 1.32 kgf/cm²g, and the operating temperature was adjusted to be 214° C.

In the case of separating phenol and hydroxyacetone using the distillation device of Comparative Example, the content of hydroxyacetone in the first bottom flow, the used amount of energy in the reboilers, the amount of reduction, the rate of reduction and the purity of the phenol product were shown in Table 1 below.

TABLE 1

| | | | | | Heat duty of reboilers | | | |
|---|---|---|---|---|---|---|---|---|
| | | | | | Total used | | | |
| | | | | Content | amount | | | |
| | | | | of HA in | of energy | | | |
| | | Input | | the first | in the | | | Purity |
| | Input | number of | Input | bottom | first and | Amount | | of |
| | temperature | theoretical | amount | flow | second | of | Rate of | phenol |
| | of AMS | stages of | of AMS | ( % by | reboilers | reduction | reduction | (% by |
| | (° C.) | AMS | (kg/hr) | weight) | (Gcal/hr) | (Gcal/hr) | (%) | weight) |
| Example 1 | 170.6 | Stage 65 | 500 | 0.131 | 16.07 | 0.72 | 4.31 | 99.99 |
| | | (bottom) | | | | | | |
| Example 2 | 170.6 | Stage 65 | 1000 | 0.051 | 16.20 | 0.60 | 3.56 | 99.99 |
| | | (bottom) | | | | | | |
| Example 3 | 24.0 | Stage 65 | 500 | 0.131 | 16.11 | 0.69 | 4.12 | 99.99 |
| | | (bottom) | | | | | | |
| Example 4 | 24.0 | Stage 65 | 1000 | 0.051 | 16.27 | 0.53 | 3.16 | 99.99 |
| | | (bottom) | | | | | | |
| Example 5 | 170.6 | Stage 61 | 500 | 0.159 | 16.23 | 0.57 | 3.40 | 99.99 |
| Example 6 | 170.6 | Stage 61 | 1000 | 0.094 | 16.06 | 0.74 | 4.42 | 99.99 |
| Example 7 | 170.6 | Stage 51 | 500 | 0.186 | 16.42 | 0.38 | 2.28 | 99.99 |
| Example 8 | 170.6 | Stage 51 | 1000 | 0.138 | 16.28 | 0.52 | 3.10 | 99.99 |
| Example 9 | 170.6 | Stage 36 | 500 | 0.203 | 16.52 | 0.28 | 1.65 | 99.99 |
| Example 10 | 170.6 | Stage 36 | 1000 | 0.164 | 16.41 | 0.39 | 2.33 | 99.99 |
| Comparative | — | — | — | 0.258 | 16.80 | — | — | — |
| Example | | | | | | | | |

1. A distillation device comprising a first distillation unit comprising a first distillation column having a first supply port and a second supply port located below the first supply port, a first condenser and a first reboiler,
wherein a feedstock comprising a first compound flows into said first supply port and a feedstock comprising a second compound forming an azeotrope with said first compound flows into said second supply port,
wherein the feedstocks comprising said first and second compound introduced into said first and second supply ports are divided into a first top flow discharged from the top region of said first distillation column and a first bottom flow discharged from the bottom region of said first distillation column, respectively, and discharged,
wherein said first top flow flows into said first condenser and some or all of the first top flow passing through said first condenser is refluxed to the top region of said first distillation column,
wherein a portion of said first bottom flow flows into said first reboiler and a portion of said first bottom flow passing through said first reboiler is refluxed to the bottom region of said first distillation column,
wherein said first top flow comprises said first compound, said second compound and a substance having a boiling point lower than that of said second compound, and said first bottom flow comprises said first compound and a substance having a boiling point higher than that of said first compound, and
wherein the content of said first compound in said first bottom flow is 0.005 to 0.25 parts by weight relative to 100 parts by weight of the total components contained in said first bottom flow.

2. The distillation device according to claim 1, wherein the content of the first compound in the first top flow is 0.01 to 2.0 parts by weight relative to 100 parts by weight of the total components contained in said first top flow.

3. The distillation device according to claim 1, wherein the first compound is hydroxyacetone.

4. The distillation device according to claim 3, wherein the second compound is alpha-methylstyrene.

5. The distillation device according to claim 3, wherein the substance having a boiling point lower than that of the second compound comprises one or more selected from the group consisting of acetone, cumene, and water.

6. The distillation device according to claim 3, wherein the substance having a boiling point higher than that of the first compound comprises one or more selected from the group consisting of cumene, phenol and methylphenyl ketone.

7. The distillation device according to claim 1, wherein the first supply port is located at 1 to 40% of the number of theoretical stages calculated on the basis of the top.

8. The distillation device according to claim 1, wherein the second supply port is located at 40 to 100% of the number of theoretical stages calculated on the basis of the top.

9. The distillation device according to claim 1, wherein a temperature of the feedstock containing the second compound introduced into the second supply port is 20 to 180° C.

10. The distillation device according to claim 1, wherein a flow rate of the feedstock containing the second compound introduced into the second supply port is 300 to 1200 kg/hr.

11. The distillation device according to claim 1, further comprising a second distillation unit comprising a second condenser, a second reboiler and a second distillation column; and a third distillation unit comprising a third condenser, a third reboiler and a third distillation column,
wherein a portion of the first bottom flow discharged from the bottom region of the first distillation column flows into said second distillation column and the flow introduced into said second distillation column is divided into a second top flow discharged from the top region of said second distillation column and a second bottom flow discharged from the bottom region of said second distillation column, respectively, and discharged,
wherein said second top flow flows into said third distillation column and the flow introduced into said third distillation column is divided into a third top flow discharged from the top region of said third distillation column and a third bottom flow discharged from the bottom region of said third distillation column, respectively, and discharged, and
wherein the content of the first compound in said third top flow is 0.01 to 5.0 parts by weight relative to 100 parts by weight of the total components contained in said third top flow.

12. The distillation device according to claim 11, wherein the third bottom flow is a flow of pure phenol.

13. A distillation method comprising a feedstock supply step of introducing a feedstock comprising a first compound into a first supply port of a first distillation column and introducing a feedstock comprising a second compound forming an azeotrope with said first compound into a second supply port located below said first supply port and located at 40 to 100% of the number of theoretical stages calculated on the basis of the top; and
a first distillation step of discharging feedstocks comprising said first and second compounds introduced into said first and second supply ports as a first top flow discharged from the top region of said first distillation column and a first bottom flow discharged from the bottom region of said first distillation column, respectively,
wherein said first top flow comprises said first compound, said second compound and a substance having a boiling point lower than that of said second compound, and said first bottom flow comprises said first compound and a substance having a boiling point higher than that of said first compound, and
wherein the content of said first compound in said first bottom flow is 0.005 to 0.25 parts by weight relative to 100 parts by weight of the total components contained in said first bottom flow.

14. The distillation method according to claim 13, wherein the content of the first compound in the first top flow is 0.01 to 2.0 parts by weight relative to 100 parts by weight of the total components contained in said first top flow.

15. The distillation method according to claim 13, comprising adjusting a temperature of the top region of the first distillation column to 89 to 107° C.

16. The distillation method according to claim 13, comprising adjusting a temperature of the bottom region of the first distillation column to 197 to 219° C.

17. The distillation method according to claim 13, comprising adjusting a pressure of the top region of the first distillation column to 0.01 to 10. kgf/cm²g.

18. The distillation method according to claim 13, comprising adjusting a pressure of the bottom region of the first distillation column to 0.5 to 1.5 kgf/cm²g.

19. The distillation process according to claim 13, wherein the first compound is hydroxyacetone and the second compound is alpha-methylstyrene.

20. The method according to claim 19, wherein the substance having a boiling point lower than that of the second compound comprises one or more selected from the group consisting of acetone, cumene and water, and the substance having a boiling point higher than that of the first compound comprises one or more selected from the group consisting of cumene, phenol and methylphenyl ketone.

21. The distillation method according to claim 13, comprising adjusting a temperature of the feedstock containing the second compound introduced into the second supply port to 20 to 180° C.

22. The distillation method according to claim 13, comprising adjusting a flow rate of the feedstock containing the second compound introduced into the second supply port to 300 to 1200 kg/hr.
